(19) United States
(12) Patent Application Publication
(10) Pub. No.: US 2003/0181885 A1
(43) Pub. Date: Sep. 25, 2003

(54) Reusable waste containment swim wear

(76) Inventor: Carole Patrice Harkness, Anderson, SC (US)

Correspondence Address: Carole P. Harkness, 204 Harborough Rd, Anderson, SC, 29625, US

(21) Appl. No.: 10/100,949

(22) Filed: Mar. 19, 2002

(51) Int. Cl. A61F 13/15; A61F 13/20

(52) U.S. Cl. 604/391; 604/386

(57) ABSTRACT

A brief for incontinent adults for containing body waste during water therapy and aquatic activities comprising a multi-layer fabric having an inner absorbent layer, impermeable central layer and a structural outer layer, said brief having Velcro side seams whereby said brief can be positioned on a human body and adjusted for snug conforming fit.

### FIELD OF THE INVENTION

This invention relates to garments which contain human waste during swimming or water therapy. More particularly, the invention relates to such garments that may be washed, cleaned, and reused after becoming soiled or they may be disposed of.

### BACKGROUND OF THE INVENTION

Aquatic therapy is useful and beneficial for many types of physical ailments and conditions and even for certain mental disorders. However, all patients and especially patients in older age groups may not be able to take advantage of aquatic therapy due to incontinence problems which occur more frequently in the older population but, incontinence can unfortunately affect patients of all ages. In addition, swimming and aquatic activities are not available to other adults who may have uncontrollable discharges such as adolescent females who have begun menstrual cycles and have not been introduced to or cannot use tampons as an alternate barrier. A problem of the prior art is that the aquatic garments available today tend to only meet the needs of infants and toddlers. An example of such a toddler garment is disclosed in U.S. Pat. No. Des. 377,980 to laid. Accordingly, one object of the present invention is to provide a garment which will adequately contain waste when submerged while worn by an adult.

Adult disposable diapers are available today under a number of brand names such as “Depends” but these diapers are not designed for swimming or other aquatic activities or therapy. Particularly, such diapers do not readily fit comfortably and effectively within close fitting, body conforming adult swim wear. Accordingly, it is another object of the present invention to provide swimming apparel for adults that fits comfortably and retain urine and fecal matter during swimming or aquatic therapy.

While a primary need for a waste containment article for aquatic activities is found with the older or geriatric segment of the population, other adults and adolescents such as those who are developmentally delayed, those with motor defects, and medical conditions do not presently have access to a suitable garment. Accordingly, it is another object of the present invention to fulfill this need and allow people of all walks of life and conditions to be able to engage in activities which require immersion in water.

A further object of the present invention is to provide a garment with the option of being washed and reused or being disposed of. Typical disposable waste containment articles are shown and described in U.S. Pat. No. 5,876,394 to Rosch et al which was granted on Mar. 2, 1999 and further disclosures can be found in the prior art cited in said Rosch et al patent.

One difficulty in the prior art disposable diapers or containment articles is that the fastening of the article to the body requires, in some instances, adhesive tape or fasteners which can be uncomfortable. Accordingly, it is another object of the present invention to provide a garment which can be readily placed and secured on an adult with a minimum of difficulty and a high degree of security and with minimum use or no use of adhesive tape.

Other objects and advantages of my invention will be readily understood and appreciated by those skilled in the art from the foregoing summary of the invention, description of the drawings, and detailed description of my invention.

### SUMMARY OF THE INVENTION

In an important aspect, my invention is a method of providing water therapy to incontinent adults comprising the steps of providing a brief of multi-layer fabric, said fabric comprising a soft, absorbent body contact layer, an impermeable layer, and a supporting or substrate fabric as the outer layer, putting the brief on in a body conforming fit, then, at least partially immersing the body of the adult in water for therapy. Preferably, the fabric comprises a central neoprene layer and a outer fabric layers bonded thereto; an inner absorbent liner or layer for contacting the body of the wearer also bonded to the neoprene. The garment has Velcro seams that allow for washing and reuse if the liner is not too heavily soiled. The Velcro seams allow for a form fit when the brief is put on. The garment or brief when laid flat before being placed on the lower body of the wearer resembles a laid out diaper.

In another aspect of my invention, I have surprisingly discovered that a waste containment article of clothing or garment can be made as a swimsuit brief with a body that comprises a multi-layer fabric with Velcro side seams that facilitate putting the garment on and removing it in the event of soiling. The Velcro seams allow for washing and reuse if the liner is not too heavily soiled. The garment when laid flat before being placed on the lower body of the wearer resembles a laid out form fitting diaper.

In another aspect, the invention comprises a reusable aquatic garment which can also be treated as a disposable garment having an outer multi-layer structure that is impermeable to fluids, and an inner body contact layer which will absorb human waste discharges. The garment is held snugly to the body of the wearer with the use of elastic around the leg openings, with Velcro side seams and optional elastic to supplement the opening at the waist. The garment may be donned normally as a panty or brief would be and then further fitted to conform body shape by adjusting the Velcro side seams. Rather than making the brief in a seamless preformed construction where the wearer “steps into” the garment, it is preferred that the garment can be readily removable by opening the side seams.

In yet another aspect, the swimsuit comprises from outside to inside an outer layer of “Lycra” and “Spandex” which are typically used in swimsuits, an impermeable layer of “Gore-Tex” brand material, french terrycloth as the absorbent layer, and swimsuit lining as the inner layer. The leg openings are gathered and will expand elastically when pulled around the legs of the wearer. The gathered area may be extended longitudinally to provide more leg contact surface to add more security against leakage. All seams that are stitched are preferably coated with Neoprene glue to further receive and waterproof them.

In a further and preferred aspect, my invention comprises a garment of panty or trunks style formed from a material having a central layer of neoprene with an outer layer and an inner layer for body contact. Such a garment is further provided with side closure means such as Velcro strips running along the sides below so that the garment will be substantially form fitting when donned.

In a still further aspect of my invention, neoprene glue can be added along all running stitches of the garments of my invention to further secure the garment while submerged.

While the garment of my invention and my method for providing water therapy are sufficient in themselves, a yet further aspect of my invention includes instructions to the patient or wearer that the garment is preferably used in conjunction with a primary absorbent article such as a sanitary napkin or urine collection pad.

My invention is better understood by reference to the drawings and detailed description which follow.

### BRIEF DESCRIPTION OF THE DRAWINGS

The drawings which are appended hereto and made a part of this disclosure are for the purpose of describing and understanding the invention and do not limit its scope. In the drawings:

FIG. 1 shows a preferred embodiment of the invention before it is shaped into a form to be worn;

FIG. 1A is a cross section view of a preferred material from which preferred embodiment can be formed;

FIG. 1B shows another embodiment of the garment of the present invention laid out as in FIG. 1 but with an alternate waist fastening means;

FIG. 2 shows a front view of the garment of FIG. 1 wherein the garment has been shaped as it would be when worn by an adult;

FIG. 3 is the back view of the garment of FIG. 2;

FIG. 4 is the front view of an alternate embodiment of my invention showing an alternate waist securing means; and,

FIG. 5 is a back view of the embodiment of FIG. 4.

### DETAILED DESCRIPTION OF A PREFERRED EMBODIMENT

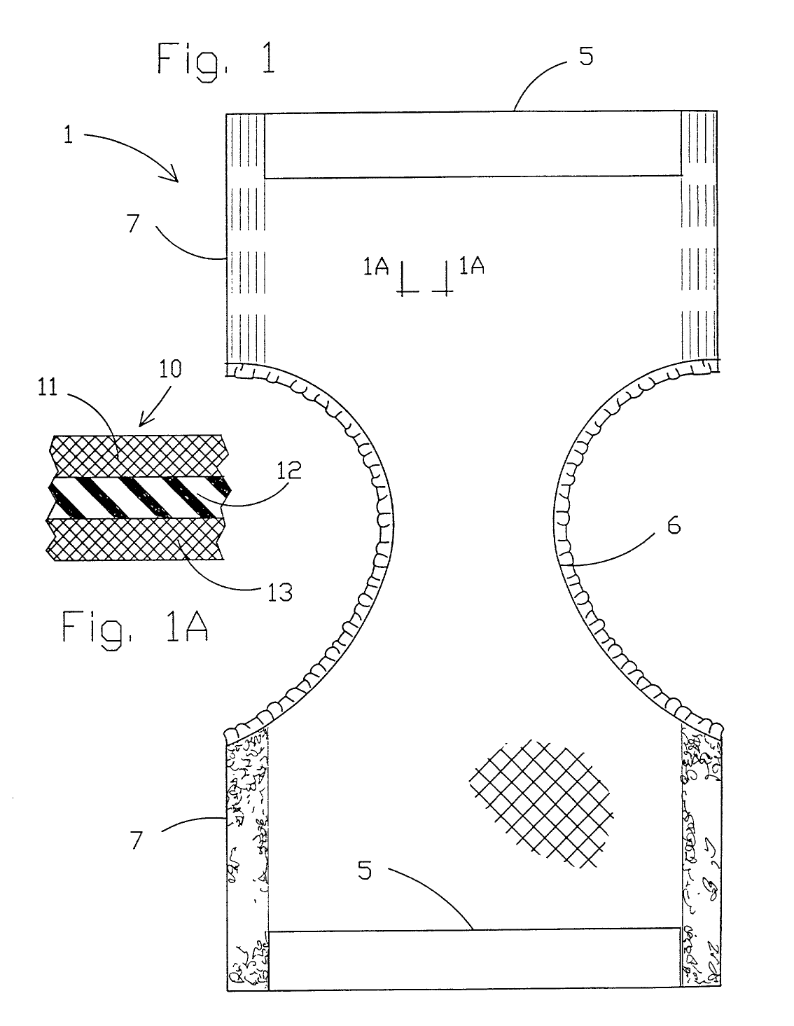

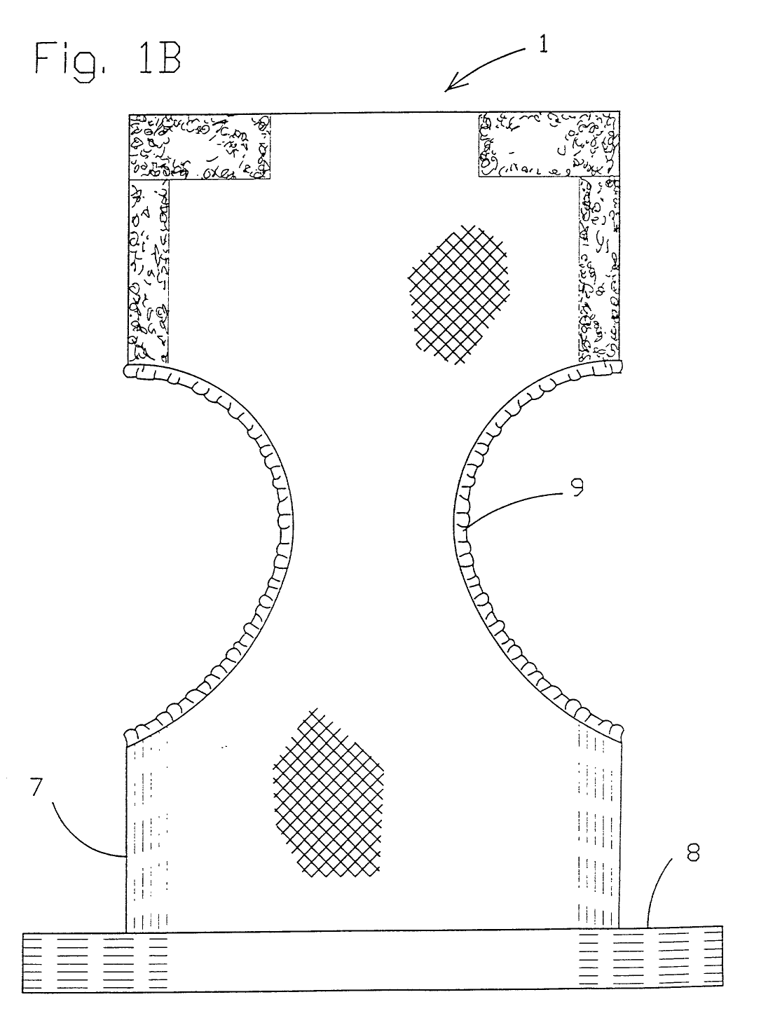

In the preferred embodiment and best mode of the present invention, the garment is made from a three layer fabric. Referring to FIGS. 1 and 1A, the arrangement of these layers may be seen. The body facing 11 comprises swimsuit lining material which is well known and used in the art and may be terrycloth, nylon, rayon, or one of these materials blended with cotton. The inner or waterproof layer 12 is preferably neoprene, or more appropriately, neoprene foam. The outer layer 13 maybe nylon, rayon or lycra. A preferred multi-layer material 10 of this construction is sold by Glomex, Inc. of Ontario, Calif. as item number SS115B130246246 NSS-1 N as a wet suit material. Other suitable fabrics are disclosed in U.S. Pat. No. 5,622,762 to Ivan Perla entitled “Wet Suit Material.” Using a fabric such as that of the Perla patent, the body contact layers are called “plush” layers and comprise cotton, silk, or synthetic fabric, the outer layer 13 is a structural material such as Lycra, and the two layers are bonded together by a coating 12 of a water resistant contact cement which functions as the waterproof layer.

In alternate material construction, the swimsuit lining 11 is the layer in contact with the body of the wearer of the garment. Proceeding outwardly and still referring to FIGS. 1 and 1A, the next layer is layer 12 which preferably comprises neoprene or other soft, cellular elastomeric material that is water proof. A woven textile material such as terry cloth with a high cotton or cellulose content for absorbency is a desirable material. A particularly useful terrycloth is one referred to as “French” terrycloth. A woven absorbent material such as terrycloth will maintain its structural and dimensioned integrity while being worn and will not tend to separate or disintegrate when exposed to body waste as will some paper or cellulose based absorbers, and can be washed and reused.

The impervious layer 12 is preferably a neoprene or water resistant bonding cements as mentioned above but also may be a woven polyester fabric pretreated with any of a number of water-repellent treatments for fabric which are well known to those skilled in the art thereby making the garment substantially leakproof. Treating a fabric with a waterproofing material such as that sold under the “Scotch Guard” brand can be done for some applications. Other material such as nylon or a soft polymeric material may be used and a preferred alternate material is that sold by Gore Association, Inc. under the “Gore-Tex” brand.

As mentioned previously, the outer layer 13 may be made of any of the well known swimsuit material such as Lycra so that the final garment has the appearance of a swimsuit.

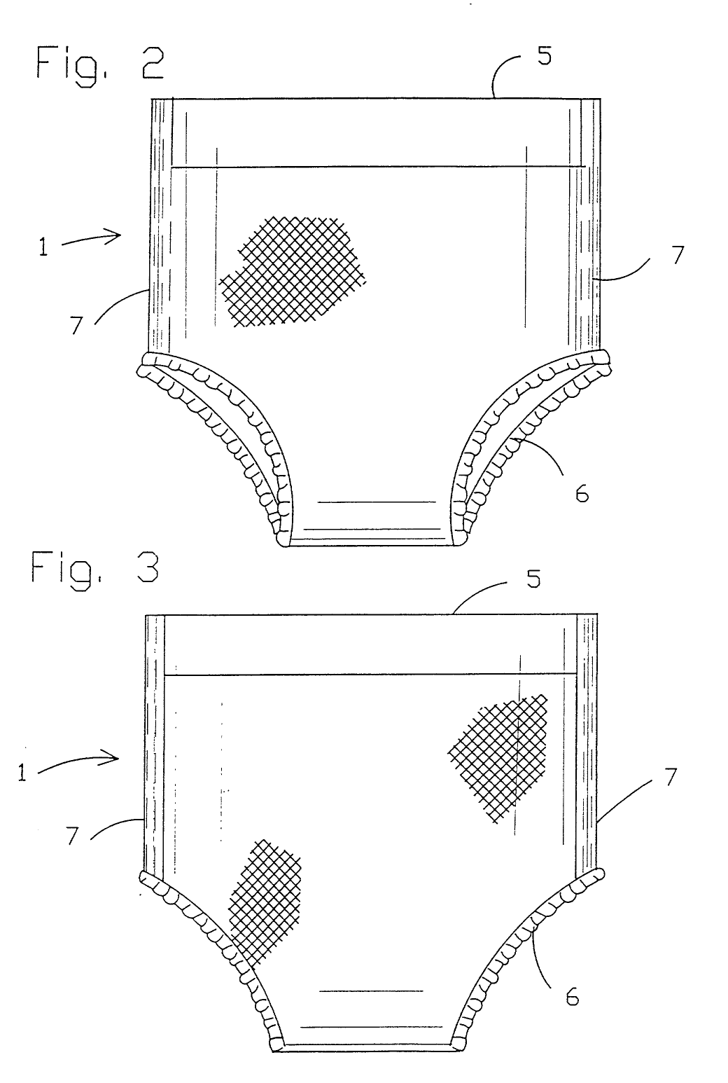

Referring now to FIGS. 2 and 3, the garment 1 of FIG. 1 is shown folded and sealed together by joining Velcro seams 7. The seams are provided at the edges of the laid out garment 1 in FIG. 1 and are sealed together as shown in FIGS. 2 and 3. The Velcro is the material which can be obtained under the brand name Velcro which is owned by Velcro B. V. of Amsterdam. The Velcro comprises one side of hooks and the other of loops so that they fasten together. One feature of the present invention is a polymeric treatment of the Velcro to enhance its integrity as a water impervious seal.

Referring to FIG. 1B, garment 1 is shown with waistband locking tabs 8 and gathered leg openings lining 6. FIG. 2 shows the leg opening with the lining completely around the opening. The gathered lining 6 may be made of any elastic material to snugly fit the leg of the wearer in the opening 9. Stitches for joining the opening elastic and waist bands are preferably covered with a neoprene glue to securely seal them against leakage.

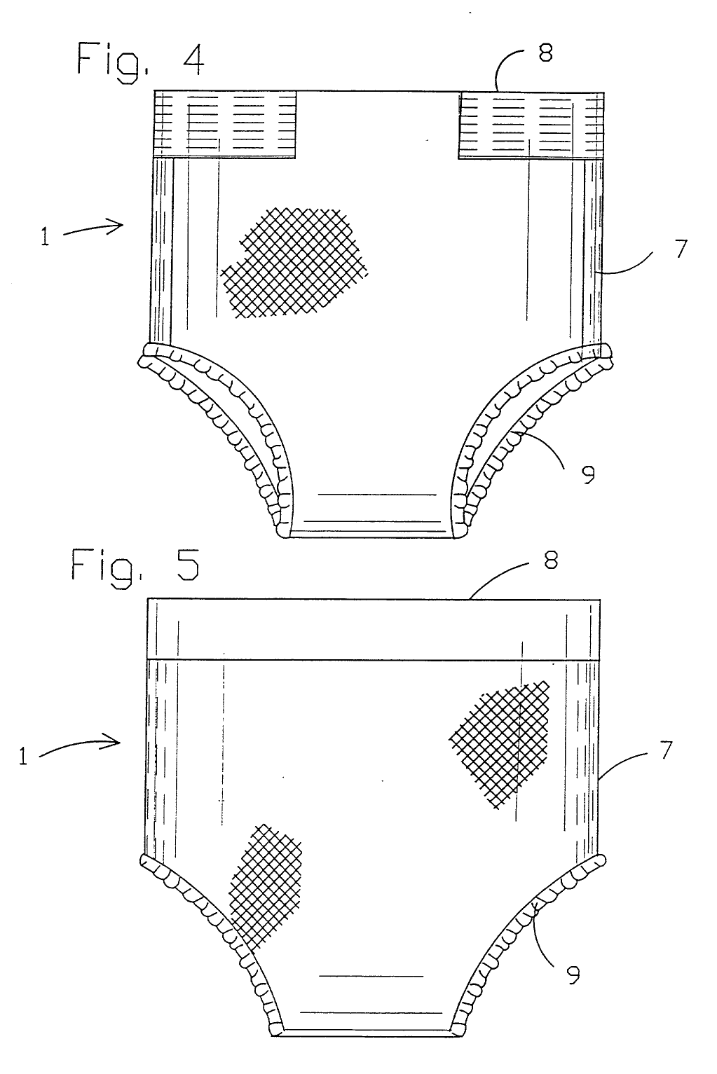

Referring now the FIGS. 2 and 3, a waistband 5 is provided which is adjustable and has mating Velcro tabs to provide a snug fit. As shown in FIGS. 1B, 4 and 5, this adjustable waistband can be about 2″ wide and it is important to cooperate with the side seams to give the garment a snug and smooth fit to the body when it is put on. This not only lessens the possibility of leakage but eliminates air pockets which can migrate to the garment margins and provide leakage sites.

Leg openings 9 are gathered at the leg hole openings and are provided with an elastic liner to prevent leakage when worn as mentioned above. The elastic liner is preferably ⅛″ to ¼″ wide but for wearers who will be very active in the water, this liner or band can be made wider to give a more effective seal between the garment and the leg. Stitching to connect the band to the garment in preferably coated with a neoprene glue.

Referring again to FIG. 1B the three layers of material represented by outer fabric layer 13, neoprene layer 12 and inner fabric layer 11 can have added thereto a disposable and/or washable layer which may be terrycloth or similar absorbent material. This disposable or washable layer by itself may comprise sublayers, namely an inner waste absorbent layer and outer permeable layer that allow waste to pass therethrough and be absorbed in the absorbent layer.

The garment of my invention described above allows adults of all ages who would otherwise be unable to participate in aquatic activities to no longer be so limited.

While preferred embodiments of the invention have been described using specific terms, such description is for illustrative purposes only, and it is to be understood that changes and variations may be made without departing from the spirit or scope of the following claims.

What I claim is:

1. A reusable waste containment swim wear brief for teenagers and adults comprising:
a) a multi-layer, diaper-shaped fabric generally rectangular in shape when laid flat, having parallel ends and sides, and opposed leg cut out openings in each side dividing each side into a first parallel pair of edges and a second parallel pair of edges.
b) said multi-layer fabric comprising an outer layer and an inner impermeable layer;
c) the first pair of side edges being provided with Velcro hooks and a second pair of side edges being provided with Velcro anchors whereby when the garment is folded and placed on a wearer, the mating Velcro edges will secure the brief on the wearer in a form fitting shape; and,
d) said inner fabric layer comprising an absorbent material.

2. The garment of claim 1 wherein the impermeable layer comprises neoprene.

3. A method whereby incontinent adults may benefit from water therapy comprising the steps of:
a) providing a multi-layer brief having a soft, absorbent inner layer, an impermeable center layer, and an outer structural layer;
b) providing Velcro side seams on said brief; and,
c) placing said brief on said adult and adjusting the Velcro side seams to a snug body conforming fit; and,
d) thereafter at least partially immersing said adult in water for therapy.
